(19) United States
(12) Patent Application Publication
(10) Pub. No.: US 2024/0293017 A1
(43) Pub. Date: Sep. 5, 2024

(54) ENDOSCOPE

(71) Applicant: FUJIFILM Corporation, Tokyo (JP)

(72) Inventors: Motoki NOKO, Kanagawa (JP); Takuro ASAOKA, Kanagawa (JP)

(73) Assignee: FUJIFILM Corporation, Tokyo (JP)

(21) Appl. No.: 18/444,738

(22) Filed: Feb. 18, 2024

(30) Foreign Application Priority Data
Mar. 2, 2023 (JP) 2023-031828

(51) Int. Cl. A61B 1/05; A61B 1/12

(52) U.S. Cl. CPC A61B 1/05; A61B 1/128

(57) ABSTRACT

An endoscope includes an imaging device that includes a lens barrel and an imaging unit, a distal end part body that has an insertion hole into which the lens barrel is inserted, and an extending part that is provided by being extended from a proximal end surface of the distal end part body, in which the imaging device includes, in a state in which the lens barrel is inserted into the insertion hole, an exposed part that is exposed from the proximal end surface of the distal end part body, and the extending part is disposed at a position facing at least the exposed part of the imaging device.

### CROSS-REFERENCE TO RELATED APPLICATION

The present application claims priority under 35 U.S.C § 119 (a) to Japanese Patent Application No. 2023-031828 filed on Mar. 2, 2023, which is hereby expressly incorporated by reference, in its entirety, into the present application.

### BACKGROUND OF THE INVENTION

### 1. Field of the Invention

The present invention relates to an endoscope, and particularly relates to an endoscope comprising an imaging device on a distal end side of an insertion part.

### 2. Description of the Related Art

A distal end part body is provided on a distal end side of the endoscope insertion part to be inserted into a body, and an imaging device including a lens barrel and an imaging unit is provided in the distal end part body.

In an endoscope having such a configuration, an imaging element constituting the imaging unit and an electronic component mounted on a substrate of the imaging element (hereinafter, referred to as a substrate including the electronic component) generate heat. For this reason, it is necessary to provide a heat dissipation structure in which the above-mentioned heat is dissipated to the outside in order to obtain a favorable image (an image of high image quality).

In JP2011-200397A, an endoscope having a heat dissipation structure in which a first highly thermally conductive resin is formed by applying a highly thermally conductive resin around each built-in component of a distal end part, and a second highly thermally conductive resin is formed by applying the highly thermally conductive resin around an outer periphery of the first highly thermally conductive resin is disclosed.

In JP1999-32985A (JP-H11-32985A), an endoscope having a heat dissipation structure in which a solid-state imaging element is fixed by a tubular spacer having high thermally conductive properties in a base part of a lens frame of an objective lens is disclosed.

In JP1986-42513U (JP-S61-42513U), an endoscope having a heat dissipation structure in which a thermally conductive material is interposed between an image sensor and a metal fitting that constitutes a distal end part is disclosed.

### SUMMARY OF THE INVENTION

By the way, the performance of an imaging device (particularly, an imaging element) in recent years has been improved. With such a high-performance imaging device, it is possible to increase the number of still images that can be captured per second, and it is possible to obtain a high-definition (high image quality) image.

However, in a case where the high-performance imaging device is mounted on an endoscope, a heat generating temperature of the imaging device also tends to be higher than that in the related art. Therefore, it is required to more effectively dissipate the heat generated by the imaging device. Since the endoscopes disclosed in JP2011-200397A, JP1999-32985A (JP-H11-32985A), and JP1986-42513U (JP-S61-42513U) do not consider the heat dissipation of the above-described high-performance imaging device, the heat of the imaging device may be accumulated inside the distal end part body, and in this case, there is a probability that an operation failure of the imaging element is caused.

The present invention has been made in consideration of such circumstances, and an object of the present invention is to provide an endoscope capable of enhancing the heat dissipation properties of a distal end part body.

In order to achieve the above object, an endoscope according to the present invention comprises an imaging device that includes a lens barrel and an imaging unit; a distal end part body that has an insertion hole into which at least the lens barrel is inserted; and an extending part that is provided by being extended from a proximal end of the distal end part body, wherein the imaging device includes, in a state in which the lens barrel is inserted into the insertion hole, an exposed part that is exposed from the proximal end of the distal end part body, and the extending part is disposed at a position facing at least the exposed part of the imaging device.

According to an aspect of the present invention, it is preferable that a space between the imaging device and the extending part may be filled with a filler having thermally conductive properties.

According to an aspect of the present invention, it is preferable that the filler may be a thermally conductive filler-mixed grease in which a grease is mixed with a thermally conductive filler.

According to an aspect of the present invention, it is preferable that the imaging unit may include an imaging element and a substrate, and the extending part faces the substrate.

According to an aspect of the present invention, it is preferable that a distal end ring that is extrapolated on a proximal end side of the distal end part body and constitutes a distal end part of a bendable part may be provided, and a heat dissipation sheet that has thermally conductive properties may be disposed on a circumference of the distal end ring, the circumference including an outer periphery on which at least the extending part is positioned.

According to the aspect of the present invention, it is preferable that the heat dissipation sheet may be disposed within a range where the extending part is positioned, in an axial direction of the distal end part body.

According to an aspect of the present invention, it is preferable that the distal end part body may have a disposition space in which the imaging unit is disposed, the disposition space being consecutively installed to the insertion hole.

According to the aspect of the present invention, it is preferable that the heat dissipation sheet may be disposed within a range where the disposition space and the extending part are positioned in an axial direction of the distal end part body.

According to an aspect of the present invention, it is preferable that the extending part may be disposed only at a position facing the exposed part of the imaging device in a circumferential direction of the distal end part body.

According to an aspect of the present invention, it is possible to enhance the heat dissipation properties of the distal end part body.

### BRIEF DESCRIPTION OF THE DRAWINGS

FIG. 1 is an overall configuration view of an endoscope of an embodiment.

FIG. 2 is an enlarged perspective view of a main part of a distal end hard part as viewed from a distal end side.

FIG. 3 is a cross-sectional view along a longitudinal axis of the distal end hard part.

FIG. 4 is a perspective view of the distal end part body as viewed from a proximal end side.

FIG. 5 is a cross-sectional view along a longitudinal axis of a distal end hard part in a modification example.

### DESCRIPTION OF THE PREFERRED EMBODIMENTS

Hereinafter, embodiments of an endoscope according to the present invention will be described with reference to the accompanying drawings.

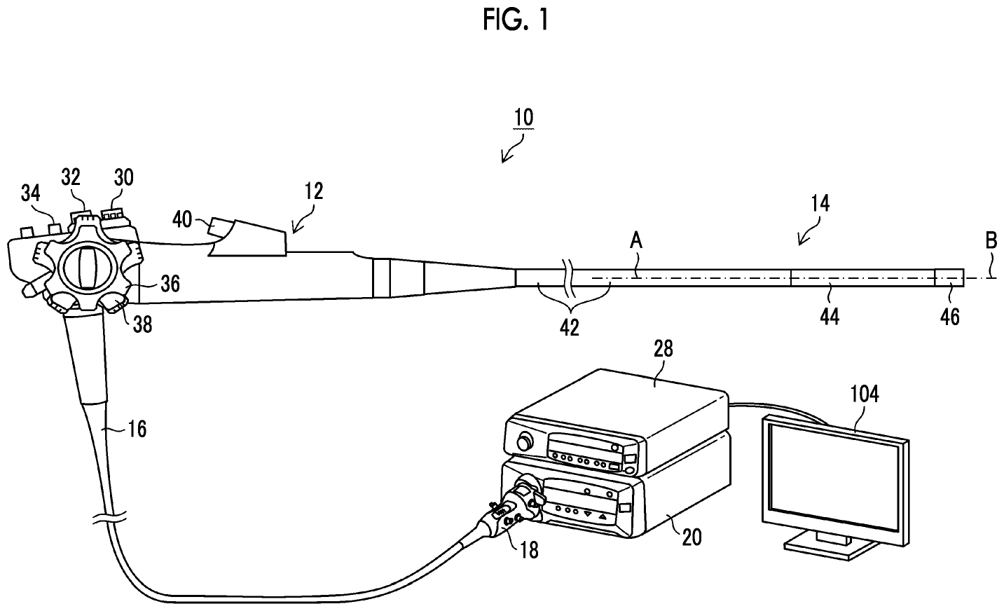

FIG. 1 is an overall configuration view of an endoscope 10 of the embodiment of the present invention. As shown in FIG. 1, the endoscope 10 comprises a hand operating part 12 that is gripped by a practitioner and an elongated insertion part 14 of which a proximal end part is connected to the hand operating part 12 and which is to be inserted into a body cavity.

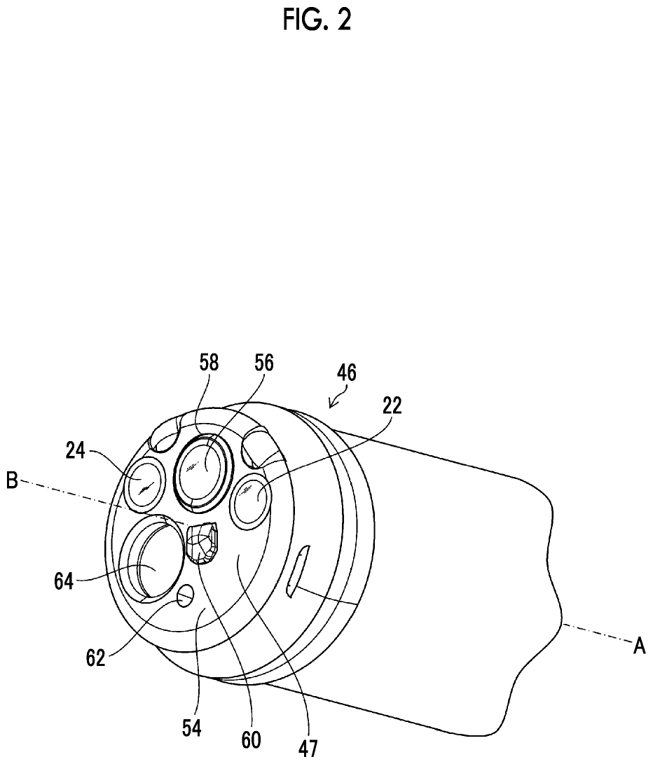

A proximal end part of a universal cable 16 is connected to the hand operating part 12, and a connector 18 is provided at a distal end part of the universal cable 16. The connector 18 is connected to a light source device 20, so that illumination light from the light source device 20 is sent to illumination windows 22 and 24 (see FIG. 2) to be described later. Further, the light source device 20 is electrically connected to the processor unit 28. The connector 18 is electrically connected to a processor unit 28 by the light source device 20. The light source device 20 and the connector 18 can transmit and receive a control signal and an image signal by optical communication. The light source device 20 transmits the control signal and the like, which is transmitted and received by optical communication via the connector 18, to the processor unit 28. The light source device 20 wirelessly supplies power for driving the endoscope 10 via the connector 18.

An air and water supply button 30, a suction button 32, and a shutter button 34 operated by a practitioner are provided in parallel in the hand operating part 12, and a pair of angle knobs 36 and 38 are rotatably provided therein. In addition, a forceps insertion part 40 for inserting a treatment tool such as a forceps is provided on a distal end side of the hand operating part 12.

The insertion part 14 has a longitudinal axis A along an insertion direction of the insertion part 14. Further, the insertion part 14 is configured such that a soft part 42, a bendable part 44, and a distal end hard part 46 are connected to each other from a proximal end part toward a distal end part of the hand operating part 12. The bendable part 44 is remotely bent by performing rotational movement operation on the pair of angle knobs 36 and 38 provided in the hand operating part 12. With this, the distal end hard part 46 can be directed in a desired direction.

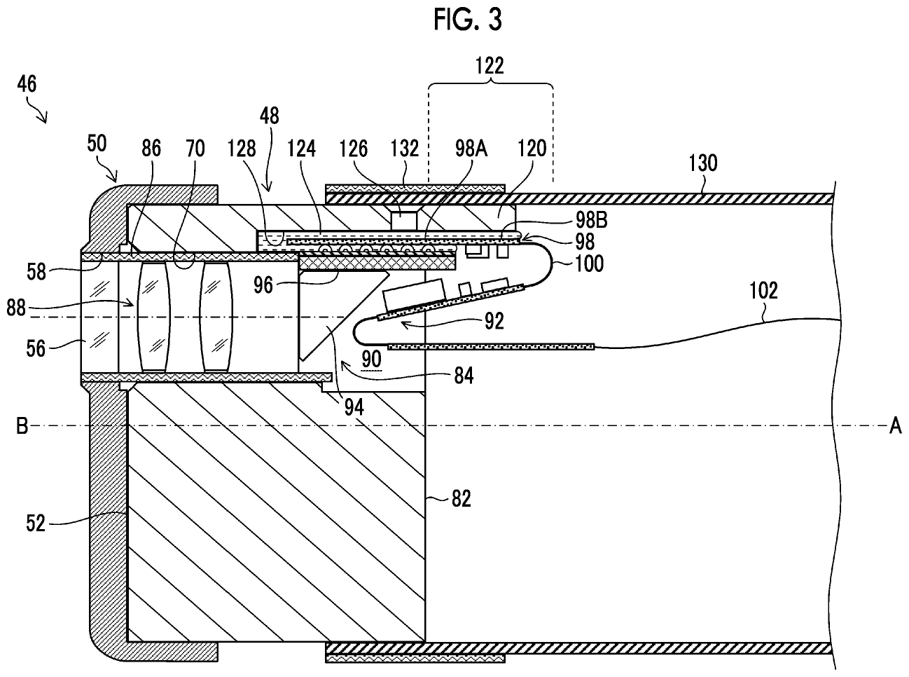

FIG. 2 is an enlarged perspective view of a main part of the distal end hard part 46 as viewed from a distal end side. FIG. 3 is a cross-sectional view of the distal end hard part 46, and is a cross-sectional view taken along the longitudinal axis A of the insertion part 14 shown in FIG. 1.

As shown in FIG. 2, a distal end surface 47 of the distal end hard part 46 is provided with an observation window 56, illumination windows 22 and 24, an air and water supply nozzle 60, a water jet port 62, and a forceps port 64.

As shown in FIG. 3, the distal end hard part 46 is configured of a distal end part body 48 provided on the distal end side of the insertion part 14, and a cap 50. For example, the distal end part body 48 is made of metal, such as stainless steel, and has a substantially columnar shape and has a central axis A-B along the longitudinal axis A. Further, the cap 50 is made of a resin and is mounted on the distal end surface 52 of the distal end part body 48. A plurality of through-holes corresponding to the observation window 56, the illumination windows 22 and 24, the air and water supply nozzle 60, the water jet port 62, and the forceps port 64 are formed in the distal end surface 54 of the cap 50.

Although not shown, the distal end part body 48 is covered with a tubular outer tube, and the distal end part of the outer tube is fixed to the distal end part body 48 by a string-like fixing member. Further, the fixing member is adhered to the outer tube by an adhesive.

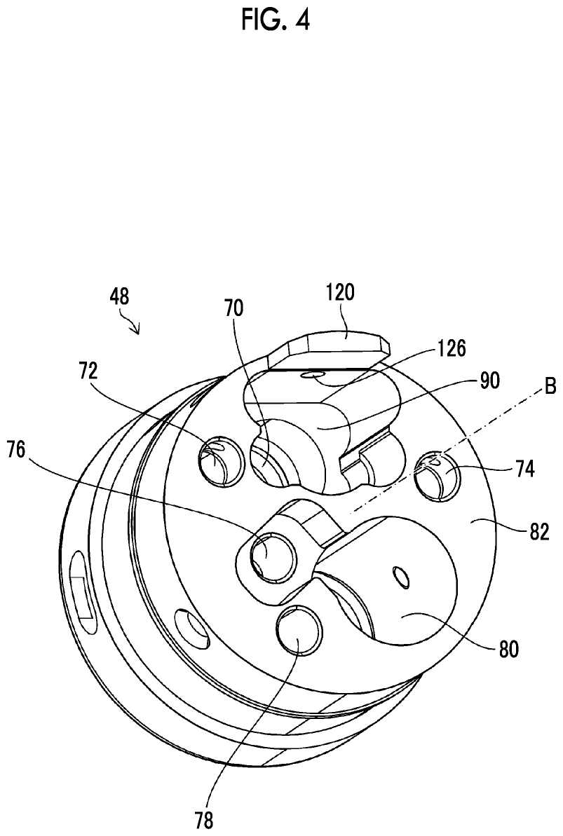

FIG. 4 is a perspective view of the distal end part body 48 as viewed from the proximal end side.

As shown in FIG. 4, circular insertion holes 70, 72, 74, 76, 78, and 80 are formed in the distal end part body 48 by being opened in a proximal end surface 82 of the distal end part body 48. These insertion holes 70 to 80 are through-holes formed from a proximal end surface 82 of the distal end part body 48 toward the distal end surface 52 of the distal end part body 48.

The insertion hole 70 of the distal end part body 48 faces a through-hole 58 of the cap 50. As shown in FIG. 3, a lens barrel 86 constituting an imaging device 84 is inserted into the insertion hole 70. The observation window 56 is fixed to a distal end side in the lens barrel 86. An optical system member 88 consisting of a plurality of lenses is fixed inside the lens barrel 86 on a proximal end side of the observation window 56. The lens barrel 86 having the above-mentioned configuration is inserted into the insertion hole 70 from a proximal end side of the distal end part body 48 along the central axis A-B, and a distal end part of the lens barrel 86 is fixed to the insertion hole 70 by being fitted into the through-hole 58 of the cap 50. The distal end part body 48 and the insertion hole 70 are an example of a distal end part body and an insertion hole of the embodiment of the present invention.

In addition, as shown in FIGS. 3 and 4, the distal end part body 48 has a disposition space 90 consecutively installed to the insertion hole 70 on the proximal end side of the insertion hole 70. An imaging unit 92 constituting the imaging device 84 is disposed in the disposition space 90. The imaging unit 92 is configured by being connected to a proximal end part of the lens barrel 86. For example, the imaging unit 92 includes a prism 94, an imaging element 96, and a substrate 98. Note that the imaging device 84, the lens barrel 86, the disposition space 90, and the imaging unit 92 are examples of an imaging device, a lens barrel, a disposition space, and an imaging unit of the embodiment of the present invention.

As shown in FIG. 3, a distal end part of a flexible cable 100 is connected to the substrate 98. A signal line 102 is connected to a proximal end part of the flexible cable 100. The signal line 102 is inserted into the insertion part 14, the hand operating part 12, and the universal cable 16 and extends up to the connector 18, and is connected to the light source device 20.

Accordingly, an observation image fetched from the observation window 56 is formed on a light-receiving surface of the imaging element 96 by the optical system member 88 of the lens barrel 86 and the prism 94. The observation image formed on the light-receiving surface of the imaging element 96 is converted into an electric signal by the imaging element 96, is output to the processor unit 28 by the signal line 102, and is converted into a video signal. As a result, the observation image is displayed on the monitor 104 connected to the processor unit 28. As the imaging element 96, a charge coupled device (CCD) image sensor or a complementary metal oxide semiconductor (CMOS) image sensor can be used.

The insertion holes 72 and 74 of the distal end part body 48 are formed on both left and right sides of the insertion hole 70. The insertion holes 72 and 74 are disposed to face two through-holes (not shown) for the illumination windows 22 and 24, which are formed in the cap 50. A distal end part of a light guide (not shown) is inserted into the insertion holes 72 and 74. A proximal end part of the light guide is inserted into the insertion part 14, the hand operating part 12, and the universal cable 16 and extends to the connector 18. Accordingly, in a case where the connector 18 is connected to the light source device 20, illumination light from the light source device 20 is transmitted to the illumination windows 22 and 24 via the light guide and is emitted forward from the illumination windows 22 and 24.

The insertion hole 76 of the distal end part body 48 is formed below the insertion hole 72. The insertion hole 76 is disposed to face a through-hole (not shown) for the air and water supply nozzle 60 formed in the cap 50. A distal end part of an air and water supply tube (not shown) is inserted into the insertion hole 76. A proximal end part of the air and water supply tube is inserted into the insertion part 14 and the hand operating part 12 and communicates with a valve (not shown) that is opened and closed by the air and water supply button 30. The valve is connected to an air and water supply connector (not shown) provided in the connector 18 by a tube (not shown). An air and water supply device (not shown) is connected to the air and water supply connector, and air or liquid is supplied from the air and water supply device to the valve through the above-described tube. Accordingly, by operating the air and water supply button 30 to open the valve, air or liquid can be jetted from the air and water supply nozzle 60 toward the observation window 56.

The insertion hole 78 of the distal end part body 48 is formed below the insertion hole 76. The insertion hole 78 is disposed to face a through-hole (not shown) for the water jet port 62 formed in the cap 50. A distal end part of a water supply tube (not shown) is inserted into the insertion hole 78. A proximal end part of the water supply tube is inserted into the insertion part 14, the hand operating part 12, and the universal cable 16, and is connected to a water supply connector (not shown) provided in the connector 18. A water supply device (not shown) having a switch is connected to the water supply connector. By driving the water supply device with the switch, a liquid is supplied from the water supply device to the water jet port 62 through the water supply tube. Accordingly, the liquid can be directly jetted from the water jet port 62 toward a portion to be tested.

An insertion hole 80 of the distal end part body 48 is formed below the central axis A-B and obliquely below the disposition space 90. The insertion hole 80 is disposed to face a through-hole (not shown) for a forceps port 64 formed in the cap 50. A distal end part of a forceps pipe (not shown) is inserted into the insertion hole 80. A distal end part of a treatment tool insertion tube (not shown) is connected to a proximal end part of the forceps pipe. A proximal end part of the treatment tool insertion tube is inserted into the insertion part 14 and communicates with the forceps insertion part 40 by a branched portion provided inside the hand operating part 12. Thus, in a case where a treatment tool such as forceps or a high-frequency scalpel is inserted from the forceps insertion part 40, the treatment tool is led out from the forceps port 64 via the treatment tool insertion tube and the forceps pipe.

Further, the branched portion communicates with a suction valve (not shown) that is opened and closed by the operation of the suction button 32 via the suction channel, and the suction valve is connected to a suction connector (not shown) provided in the connector 18, through a tube (not shown). Accordingly, by connecting a suction pump (not shown) to the suction connector and operating the suction button 32 to open the suction valve, residues, filth, or the like from the forceps port 64 can be sucked through the forceps pipe, the treatment tool insertion tube, and the suction channel.

Next, a heat dissipation structure in the endoscope 10 of the embodiment will be described.

As shown in FIGS. 3 and 4, the distal end part body 48 includes an extending part 120. The extending part 120 is provided to extend from a proximal end surface 82 of the distal end part body 48. Specifically, the extending part 120 is provided to extend to the proximal end side of the insertion part 14 from an outer peripheral portion of the proximal end surface 82.

The imaging device 84 includes an exposed part 122 that is exposed from the proximal end surface 82 of the distal end part body 48 in a state in which the lens barrel 86 is inserted into the insertion hole 70. The extending part 120 is disposed outward of the exposed part 122 with respect to the central axis A-B, and is disposed at a position facing the exposed part 122 in the radial direction orthogonal to the central axis A-B. The extending part 120 is an example of an extending part of the embodiment of the present invention.

Here, the exposed part 122 is a portion of the imaging unit 92 that is disposed closer to the proximal end side of the insertion part 14 than the proximal end surface 82 of the distal end part body 48 in a state in which the imaging unit 92 is disposed in the disposition space 90. In the present example, a portion of the substrate 98 constituting the imaging unit 92 that is disposed from the proximal end surface 82 to the proximal end side of the insertion part 14 is the exposed part 122. In other words, a portion (hereinafter, referred to as a substrate-proximal end-side portion) 98B extending toward the proximal end side from a substantially central part 98A between the distal end and the proximal end of the substrate 98 in a direction of the central axis A-B is the exposed part 122. The exposed part 122 is an example of an exposed part of the embodiment of the present invention. In the following description, the exposed part 122 may be referred to as a substrate-proximal end-side portion 98B.

Therefore, the extending part 120 of this example is disposed to face the exposed part 122 (that is, the substrate-proximal end-side portion 98B) of the imaging device 84. Further, the extending part 120 is disposed only at a position facing the substrate-proximal end-side portion 98B in the circumferential direction of the distal end part body 48.

As shown in FIG. 3, a space between the imaging device 84 and the extending part 120 is filled with a filler 124 having thermally conductive properties. In the embodiment, as the filler 124, a grease in which a thermally conductive filler such as an alumina powder is mixed with a silicone grease is used in the present example. The filler 124 and the grease are examples of a filler and thermally conductive filler-mixed grease of the embodiment of the present invention.

The distal end part body 48 has a through-hole 126 for filling the distal end part body 48 with a filler 124. The through-hole 126 is a hole that allows an outer peripheral surface of the distal end part body 48 to communicate with the disposition space 90. A gap between the distal end part body 48 and the substrate 98 (specifically, a gap between an interior wall 128 forming the disposition space 90 and the substrate 98 (excluding the substrate-proximal end-side portion 98B), and a gap between the extending part 120 and the substrate-proximal end-side portion 98B) is filled with the filler 124 injected from the through-hole 126.

As shown in FIG. 3, a distal end ring 130 constituting the distal end part of the bendable part 44 (see FIG. 1) is extrapolated on a proximal end side of the distal end part body 48. A heat dissipation sheet 132 is disposed on an outer peripheral surface of the distal end ring 130. The heat dissipation sheet 132 is disposed on a circumference including an outer periphery on which at least the extending part 120 of the distal end ring 130 is positioned.

Specifically, the heat dissipation sheet 132 is disposed (rolled) in a range where the disposition space 90 and the extending part 120 are positioned in the direction of the central axis A-B of the distal end part body 48. In the present example, since the substrate 98, which is the heat generation source, is disposed in the disposition space 90 formed in the distal end part body 48, the heat dissipation sheet 132 is disposed not only on the outer periphery on which the extending part 120 is positioned, but also in a range where the disposition space 90 and the extending part 120 are positioned in order to effectively dissipate heat of the entire substrate 98.

As the heat dissipation sheet 132, a graphite sheet is applied in the present example. The graphite sheet is a thin graphite sheet having both flexibility and heat dissipation properties and has a high thermal conductivity. The heat dissipation sheet 132 is an example of a heat dissipation sheet according to the embodiment of the present invention.

Next, the effects of the endoscope 10 according to the embodiment will be described.

The endoscope 10 according to the embodiment has a heat dissipation structure in which the extending part 120 extending from the proximal end surface 82 of the distal end part body 48 is disposed at a position facing the exposed part 122 (substrate-proximal end-side portion 98B). Therefore, the heat generated in the exposed part 122 of the substrate 98 can be effectively received by the extending part 120. Accordingly, the heat generated in the exposed part 122 is transferred and diffused from the extending part 120 to the entire distal end part body 48 (including the extending part 120) without being accumulated in the vicinity of the exposed part 122, and the heat is radiated from the entire distal end part body 48. Therefore, the heat dissipation properties of the distal end part body 48 can be enhanced.

In addition, according to the endoscope 10 of the embodiment, since the space between the imaging device 84 and the extending part 120 is filled with the filler 124 having thermally conductive properties, the heat generated in the entire substrate 98 (including the substrate-proximal end-side portion 98B) can be effectively transferred to the entire distal end part body 48.

In addition, according to the endoscope 10 of the embodiment, since the heat dissipation sheet 132 is disposed on the outer periphery of the distal end ring 130 on which at least the extending part 120 is positioned, the heat of the exposed part 122 received by the extending part 120 can be diffused by the heat dissipation sheet 132. The heat diffused by the heat dissipation sheet 132 is further diffused and released from the bendable part 44 to the soft part 42 and the like through the structure of the endoscope 10. Accordingly, the heat of the exposed part 122 can be radiated more effectively.

In addition, it is preferable that the above-described heat dissipation sheet 132 is disposed in a range where the disposition space 90 and the extending part 120 are positioned in the direction of the central axis A-B of the distal end part body 48. The heat of the substrate 98 diffused by the distal end part body 48 (including the extending part 120) is diffused by the heat dissipation sheet 132 and is released through the above-described route (the structure of the endoscope, the bendable part 44, and the soft part 42). Accordingly, the heat of the entire substrate 98 can be effectively radiated.

Therefore, according to the endoscope 10 of the embodiment, the heat generated in the substrate 98 can be effectively radiated to the outside of the endoscope 10. Therefore, even a high-performance imaging device having a high heat generation temperature, can be mounted.

Hereinafter, modification examples of the endoscope of the embodiment of the present invention will be described.

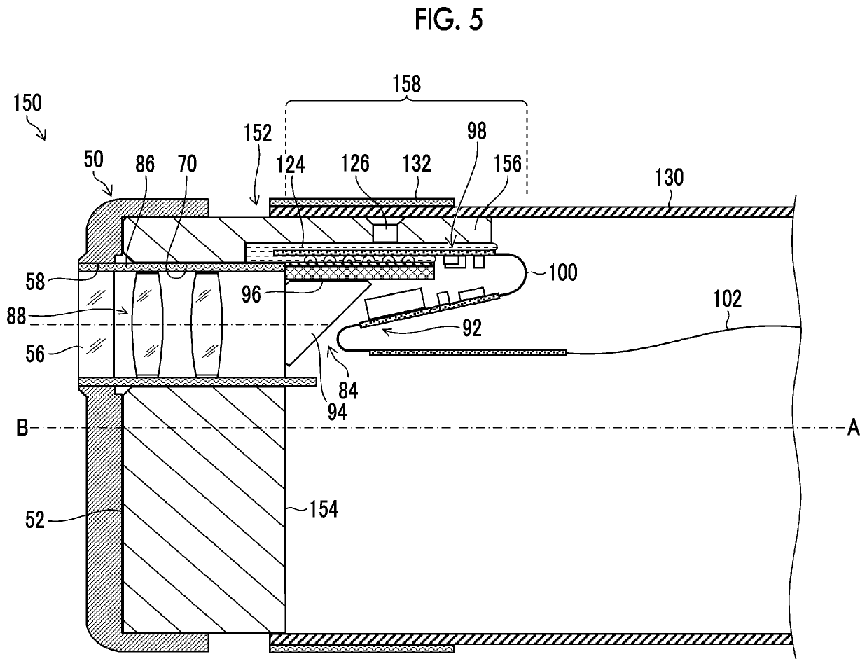

FIG. 5 is a cross-sectional view along the longitudinal axis A of a distal end hard part 150 in a modification example. In the description of the distal end hard part 150 shown in FIG. 5, the same or similar members as or to those of the distal end hard part 46 of the above-described embodiment will be denoted by the same reference numerals.

The difference between the modification example shown in FIG. 5 and the above-described embodiment is that, in the above-described embodiment, the distal end part body 48 of the distal end hard part 46 includes the disposition space 90 as a space for disposing the imaging unit 92, whereas in the modification example shown in FIG. 5, the distal end part body 152 of the distal end hard part 150 does not have a space for disposing the imaging unit 92.

As shown in FIG. 5, the distal end part body 152 of the modification example is configured to be shorter in length in the direction of the central axis A-B than the distal end part body 48 shown in FIG. 3 by an amount of not having a space for disposing the imaging unit 92. An extending part 156 is provided to extend from a proximal end surface 154 of the distal end part body 152. The extending part 156 is configured to have a longer length in the direction of the central axis A-B than the extending part 120 (see FIG. 3) in the above-described embodiment, by an amount of not having a space for disposing the imaging unit 92.

The imaging device 84 includes an exposed part 158 that is exposed from the proximal end surface 154 of the distal end part body 152 in a state in which the lens barrel 86 is inserted into the insertion hole 70. The extending part 156 is disposed at a position facing the exposed part 158. Note that, in the distal end part body 152 of the modification example, the exposed part 158 corresponds to the imaging unit 92 that is exposed from the proximal end surface 154 of the distal end part body 152.

Further, in the distal end part body 152, a space between the substrate 98 and the extending part 156 is filled with the filler 124.

In addition, the heat dissipation sheet 132 is disposed on a circumference including an outer periphery on which at least the extending part 156 of the distal end ring 130 is positioned. Specifically, the heat dissipation sheet 132 is disposed in a range where the extending part 156 is positioned in the direction of the central axis A-B of the distal end part body 48.

Even in the modification example configured as described above, as in the above-described embodiment, heat generated in the substrate 98 can be effectively dissipated to the outside of the endoscope 10 by using the filler 124, the extending part 156, and the heat dissipation sheet 132.

In the embodiment, an example in which the extending part 120 is disposed only at a position facing the exposed part 122 in the circumferential direction of the distal end part body 48 has been described, but the present invention is not limited thereto. That is, the extending part 120 may be disposed at a position facing at least the exposed part 122. For example, the length of the extending part 120 may be configured to be longer than the length of the exposed part 122 in the circumferential direction of the distal end part body 48 in a case where the proximal end surface 82 of the distal end part body 48 is viewed from the proximal end side.

In the embodiment, an example in which the filler 124 is used has been described, but the present invention is not limited thereto. For example, the substrate 98 and the distal end part body 48 (the interior wall 128 of the disposition space 90 and the extending part 120) may be disposed in contact with or close to each other without using the filler 124. Accordingly, heat generated in the substrate 98 can be transferred to the distal end part body 48. However, from the viewpoint of effectively transferring heat generated in the substrate 98 to the distal end part body 48, it is preferable to use the filler 124.

Although the endoscope according to the embodiment is described above, the present invention may be improved or modified in some ways without departing from the gist of the present invention.

### EXPLANATION OF REFERENCES

10: endoscope
12: hand operating part
14: insertion part
16: universal cable
18: connector
20: light source device
22: illumination window
24: illumination window
28: processor unit
30: air and water supply button
32: suction button
34: shutter button
36: angle knob
38: angle knob
40: forceps insertion part
42: soft part
44: bendable part
46: distal end hard part
47: distal end surface
48: distal end part body
50: cap
52: distal end surface
54: distal end surface
56: observation window
58: through-hole
60: air and water supply nozzle
62: water jet port
64: forceps port
70: insertion hole
72: insertion hole
74: insertion hole
76: insertion hole
78: insertion hole
80: insertion hole
82: proximal end surface
84: imaging device
86: lens barrel
88: optical system member
90: disposition space
92: imaging unit
94: prism
96: imaging element
98: substrate
98A: substantially central part
98B: substrate-proximal end-side portion
100: flexible cable
102: signal line
104: monitor
120: extending part
122: exposed part
124: filler
126: through-hole
130: distal end ring
132: heat dissipation sheet
150: distal end hard part
152: distal end part body
154: proximal end surface
156: extending part
158: exposed part

What is claimed is:

1. An endoscope comprising:
an imaging device that includes a lens barrel and an imaging unit;
a distal end part body that has an insertion hole into which at least the lens barrel is inserted; and
an extending part that is provided by being extended from a proximal end of the distal end part body,
wherein the imaging device includes, in a state in which the lens barrel is inserted into the insertion hole, an exposed part that is exposed from the proximal end of the distal end part body, and
the extending part is disposed at a position facing at least the exposed part of the imaging device.

2. The endoscope according to claim 1,
wherein a space between the imaging device and the extending part is filled with a filler having thermally conductive properties.

3. The endoscope according to claim 2,
wherein the filler is a thermally conductive filler-mixed grease in which a grease is mixed with a thermally conductive filler.

4. The endoscope according to claim 1,
wherein the imaging unit includes an imaging element and a substrate, and
the extending part faces the substrate.

5. The endoscope according to claim 4,
wherein a distal end ring that is extrapolated on a proximal end side of the distal end part body and constitutes a distal end part of a bendable part is provided, and
a heat dissipation sheet that has thermally conductive properties is disposed on a circumference of the distal end ring, the circumference including an outer periphery on which at least the extending part is positioned.

6. The endoscope according to claim 5,
wherein the heat dissipation sheet is disposed within a range where the extending part is positioned, in an axial direction of the distal end part body.

7. The endoscope according to claim 5,
wherein the distal end part body has a disposition space in which the imaging unit is disposed, the disposition space being consecutively installed to the insertion hole.

8. The endoscope according to claim 7,
wherein the heat dissipation sheet is disposed within a range where the disposition space and the extending part are positioned in an axial direction of the distal end part body.

9. The endoscope according to claim 1,
wherein the extending part is disposed only at a position facing the exposed part of the imaging device in a circumferential direction of the distal end part body.
